(19) United States
(12) Patent Application Publication
(10) Pub. No.: US 2022/0372570 A1
(43) Pub. Date: Nov. 24, 2022

(54) CONTROLLED CROSSLINKING OF BIOMOLECUES IN SITU

(71) Applicant: 10x Genomics, Inc., Pleasanton, CA (US)

(72) Inventor: Justin COSTA, Union City, CA (US)

(21) Appl. No.: 17/738,538

(22) Filed: May 6, 2022

Related U.S. Application Data
(60) Provisional application No. 63/185,944, filed on May 7, 2021.

(51) Int. Cl. C12Q 1/6876; C12Q 1/682

(52) U.S. Cl. CPC C12Q 1/6876; C12Q 1/682

(57) ABSTRACT

The present disclosure relates in some aspects to methods for analyzing a target nucleic acid in a biological sample. In some aspects, the methods involve the use of a set of oligonucleotides, for example a set of two or more oligonucleotides, wherein one or more oligonucleotides comprises one or more photoreactive nucleotides, for analyzing target nucleic acids. In some aspects, the presence, amount, and/or identity of a target nucleic acid is analyzed in situ. Also provided are oligonucleotides, sets of oligonucleotides, compositions, and kits for use in accordance with the methods.

### CROSS-REFERENCE TO RELATED APPLICATIONS

This application claims priority to U.S. Provisional Patent Application No. 63/185,944, filed May 7, 2021, entitled “Controlled Crosslinking of Biomolecules In Situ,” which is herein incorporated by reference in its entirety for all purposes.

### FIELD

The present disclosure relates in some aspects to methods and compositions for analysis of a target nucleic acid in a sample (e.g., in situ), such as analysis using oligonucleotides comprising crosslinkable nucleotides.

### BACKGROUND

Oligonucleotide probe-based assay methods for analysis of target nucleic acids depend on careful optimization related to the stability of the hybridization complex and/or the positional stability of the hybridization complex. For example, if the wash conditions are too stringent, then probe/target hybrids or amplification products thereof will be denatured, resulting in a decrease in the amount of signal in the assay. Furthermore, some methods such as isometric expansion of a sample require stabilization of target analytes to a matrix in order to preserve positional information of the target analytes in the sample (e.g., a cell or tissue sample). Thus, there is a need for increasing the spatial fidelity of target analytes (e.g., present in amplification products, such as rolling circle amplification products) during analysis of target nucleic acids in a sample (e.g., in situ analysis). Provided herein are methods and compositions that address such and other needs.

### BRIEF SUMMARY

Provided herein are methods and compositions for controlled crosslinking of biomolecules in situ in a biological sample, including methods for enhancing the spatial fidelity of nucleic acid molecules (such as nucleic acid concatemers) during in situ analysis, e.g., for decoding nucleic acid barcode sequences through sequential cycles of probe hybridization. In some aspects, provided herein is a method for processing a biological sample, e.g., for sequential hybridization of detectable probes to a nucleic acid molecule in the biological sample. In some embodiments, the method comprises: (a) generating a nucleic acid concatemer in the biological sample, wherein the nucleic acid concatemer comprises a photoreactive nucleotide in a hybridization region hybridized to a complementary strand; and (b) photo-activating the photoreactive nucleotide to react with a nucleotide in the complementary strand, thereby crosslinking the nucleic acid concatemer to the complementary strand.

In any of the preceding embodiments, the nucleic acid concatemer can be or comprise a product of an analyte in the biological sample. In any of the preceding embodiments, the analyte may be a nucleic acid analyte.

In any of the preceding embodiments, the nucleic acid concatemer can be or comprise a product of a labeling agent that directly or indirectly binds to an analyte in the biological sample. In any of the preceding embodiments, the analyte may be a nucleic acid analyte or a non-nucleic acid analyte. In any of the preceding embodiments, the labeling agent can comprise a reporter oligonucleotide and an analyte-binding moiety coupled thereto.

In any of the preceding embodiments, the nucleic acid concatemer can be or comprise a rolling circle amplification product. In any of the preceding embodiments, the nucleic acid concatemer may comprise one or more barcode sequences. In any of the preceding embodiments, the nucleic acid concatemer may comprise a barcode sequence corresponding to an analyte in the biological sample.

In any of the preceding embodiments, the nucleic acid concatemer can be between about 1 and about 15 kilobases, between about 15 and about 25 kilobases, between about 25 and about 35 kilobases, between about 35 and about 45 kilobases, between about 45 and about 55 kilobases, between about 55 and about 65 kilobases, between about 65 and about 75 kilobases, or more than 75 kilobases in length. In any of the preceding embodiments, the nucleic acid concatemer may form a nanoball having a diameter between about 0.1 μm and about 3 μm. In any of the preceding embodiments, the nucleic acid concatemer may comprise one or more amine-modified nucleotides, for example, for crosslinking the nucleic acid concatemer to a matrix embedding the biological sample. The one or more amine-modified nucleotides may be incorporated during a reaction (e.g., rolling circle amplification) for generating or processing the nucleic acid concatemer.

In any of the preceding embodiments, the photoreactive nucleotide may be a modified nucleotide comprising a psoralen or a psoralen derivative. In some embodiments, the photoreactive nucleotide may comprise a psoralen or a psoralen derivative connected to a nucleotide via a linker. In some embodiments, the photoreactive nucleotide may be a modified nucleotide comprising a vinylcarbazone-based moiety. In some embodiments, the modified nucleotide may comprise a 3-cyanovinylcarbazole (^{CNV}K) nucleoside, 3-cyanovinylcarbazole modified D-threoninol (^{CNV}D), a pyranocarbazole (^{PC}X) nucleoside, or a pyranocarbazole modified D-threoninol (^{PCX}D). In some embodiments, the photoreactive nucleotide may comprise a 3-cyanovinylcarbazole phosphoramidite or a pyranocarbazole phosphoramidite. In some embodiments, the photoreactive nucleotide may comprise a vinylcarbazone-based moiety connected to a nucleotide via a linker.

In any of the preceding embodiments, the nucleic acid concatemer may comprise two or more photoreactive nucleotides in the hybridization region. In any of the preceding embodiments, the nucleic acid concatemer may comprise three, four, five or more photoreactive nucleotides in the hybridization region. Any of the photoreactive nucleotides can be ^{CNV}D, ^{CNV}K, or any other suitable photoreactive nucleotides.

In any of the preceding embodiments, the hybridization region may comprise a 5′ end sequence, an internal sequence, and/or a 3′ end sequence of the nucleic acid concatemer. In any of the preceding embodiments, the hybridization region may be 5′ to the tandem unit sequences of the nucleic acid concatemer. In any of the preceding embodiments, the hybridization region may be at the 5′ end of the nucleic acid concatemer.

In any of the preceding embodiments, the hybridization region may be between about 5 and about 30 nucleotides in length. In any of the preceding embodiments, the hybridization region may be between about 10 and about 15 nucleotides in length.

In any of the preceding embodiments, the complementary strand may be coupled to a matrix embedding the biological sample. In any of the preceding embodiments, the complementary strand may be crosslinked in the biological sample before the generating in step (a) in which the nucleic acid concatemer is generated.

In any of the preceding embodiments, the complementary strand may be in a cellular RNA in the biological sample. In any of the preceding embodiments, the complementary strand may be in an mRNA crosslinked in the biological sample before the generating in step (a). In some embodiments, the crosslinking can be formaldehyde crosslinking. In any of the preceding embodiments, the complementary strand may be in a cDNA in the biological sample. In any of the preceding embodiments, the complementary strand may be in a genomic DNA in the biological sample. In any of the preceding embodiments, the complementary strand may be crosslinked in the biological sample before the nucleic acid concatemer is generated.

In any of the preceding embodiments, the method can further comprise providing an oligonucleotide comprising the hybridization region before the nucleic acid concatemer is generated in step (a), wherein the oligonucleotide hybridizes to the complementary strand. In any of the preceding embodiments, the method can further comprise providing a circular probe or circularizable probe or probe set before the nucleic acid concatemer is generated step (a), wherein the oligonucleotide and the circular probe or circularizable probe or probe set hybridize to adjacent regions in the complementary strand. In any of the preceding embodiments, the oligonucleotide can further comprise a primer region that hybridizes to the circular probe or circularizable probe or probe set, whereby the nucleic acid concatemer is generated using the oligonucleotide as a primer and the circular probe or circularizable probe or probe set as a template. In any of the preceding embodiments, the method can further comprise ligating the oligonucleotide to a primer that hybridizes to the circular probe or circularizable probe or probe set, whereby the nucleic acid concatemer is generated using the primer as a primer and the circular probe or circularizable probe or probe set as a template. In any of the preceding embodiments, the method can further comprise removing the oligonucleotide, the circular probe or circularizable probe or probe set, and/or the primer that are not stably hybridized to the complementary strand and/or to one another. In any of the preceding embodiments, the method can further comprise washing the biological sample under a stringent condition. In any of the preceding embodiments, the method can further comprise ligating the circularizable probe or probe set to generate a circularized template for rolling circle amplification to generate the nucleic acid concatemer.

In any of the preceding embodiments, the oligonucleotide can comprises one or more universal bases. In some embodiments, the one or more universal bases are in the hybridization region of the oligonucleotide. In any of the preceding embodiments, the photoreactive nucleotide can comprise the universal base. In any of the preceding embodiments, the oligonucleotide can comprise two or more photoreactive nucleotides in the hybridization region, and the photoreactive nucleotides can comprise the universal bases. In any of the preceding embodiments, the one or more universal bases can comprise a pseudouridine and/or an inosine. In some embodiments, the one or more universal bases comprise pseudouridine. In any of the preceding embodiments, the hybridization region of the oligonucleotide can comprise a plurality of universal bases. In any of the preceding embodiments, the hybridization region of the oligonucleotide may hybridize to the complementary strand non-specifically.

In any of the preceding embodiments, the photo-activating in step (b) may be performed using a 350-400 nm wavelength of light. In any of the preceding embodiments, the photo-activating in step (b) may be performed after the nucleic acid concatemer is generated in step (a). In any of the preceding embodiments, the photo-activating in step (b) may be performed before the nucleic acid concatemer is generated in step (a). In any of the preceding embodiments, the photo-activating in step (b) may be performed as the nucleic acid concatemer is being generated in step (a).

In any of the preceding embodiments, crosslinking the nucleic acid concatemer to the complementary strand may increase the UV melting temperature of the duplex compared to prior to the crosslinking. In any of the preceding embodiments, the UV melting temperature may be increased by about 30° C. per photoreactive nucleotide in the hybridization region.

In any of the preceding embodiments, the method may further comprise (c) processing the biological sample under a denaturing condition. In any of the preceding embodiments, the processing of the biological sample under a denaturing condition, e.g., in (c), may comprise contacting the biological sample with a denaturing agent and/or heating the biological sample. In any of the preceding embodiments, the denaturing agent may be formamide. In any of the preceding embodiments, the nucleic acid concatemer crosslinked to the complementary strand may remain associated with the complementary strand, whereas a reference nucleic acid concatemer not comprising the photoreactive nucleotide may be dissociated from the complementary strand.

In any of the preceding embodiments, the method may further comprise detecting the nucleic acid concatemer in the biological sample, e.g., using fluorescent microscopy for detecting the nucleic acid concatemer in situ.

In any of the preceding embodiments, the detecting may comprise contacting the biological sample with one or more detectably-labeled probes that directly or indirectly hybridize to the nucleic acid concatemer. In any of the preceding embodiments, the detecting may further comprise dehybridizing the one or more detectably-labeled probes from the nucleic acid concatemer, e.g., by contacting the biological sample with a denaturing agent and/or heating the biological sample. In any of the preceding embodiments, the detecting may further comprise repeating the contacting and dehybridizing steps with the one or more detectably-labeled probes and/or one or more other detectably-labeled probes that directly or indirectly hybridize to the nucleic acid concatemer.

In any of the preceding embodiments, the detecting may comprise contacting the biological sample with one or more intermediate probes that directly or indirectly hybridize to the nucleic acid concatemer, wherein the one or more intermediate probes are detectable using one or more detectably-labeled probes. In any of the preceding embodiments, the detecting may further comprise dehybridizing the one or more intermediate probes and/or the one or more detectably-labeled probes from the nucleic acid concatemer. In any of the preceding embodiments, the detecting may further comprise repeating the contacting and dehybridizing steps with the one or more intermediate probes, the one or more detectably-labeled probes, one or more other intermediate probes, and/or one or more other detectably-labeled probes.

In any of the preceding embodiments, the photoreactive nucleotide and/or the nucleotide in the complementary strand that have reacted with each other may remain in the nucleic acid concatemer or the complementary strand, respectively, and may not need to be removed (e.g., by photo-cleavage, chemical cleavage, and/or enzymatic cleavage) prior to detecting the nucleic acid concatemer in the biological sample.

In some aspects, provided herein is a method for analyzing a biological sample, comprising: (a) contacting the biological sample with: a circular probe or circularizable probe or probe set, and an oligonucleotide comprising (i) a hybridization region that hybridizes to a target nucleic acid in the biological sample and (ii) a primer sequence that hybridizes to the circular probe or circularizable probe or probe set, wherein the hybridization region comprises a photoreactive nucleotide; (b) photo-activating the photoreactive nucleotide to react with a nucleotide in the target nucleic acid, thereby crosslinking the oligonucleotide to the target nucleic acid; (c) generating a rolling circle amplification (RCA) product in the biological sample, using the primer sequence as a primer and the circular probe or a circularized probe generated from the circularizable probe or probe set as a template, wherein the RCA product comprises the oligonucleotide or a portion thereof crosslinked to the target nucleic acid; (d) contacting the biological sample with a detection probe that hybridizes to the RCA product; and (e) dehybridizing the detection probe from the RCA product while the RCA product remains crosslinked to the target nucleic acid in the biological sample. In some embodiments, the method can further comprise contacting the sample with a subsequent detection probe that hybridizes to the RCA product and dehybridizing the subsequent detection probe from the RCA product while the RCA product remains crosslinked to the target nucleic acid in the biological sample.

In some aspects, provided herein is a method for analyzing a biological sample, comprising: (a) contacting the biological sample with: a first circular probe or circularizable probe or probe set that hybridizes to a first target sequence in a first complementary strand, a second circular probe or circularizable probe or probe set that hybridizes to a second target sequence in a second complementary strand, and a first and second oligonucleotide, wherein each oligonucleotide comprises (i) a common hybridization region comprising one or more photoreactive nucleotide comprising a universal base and (ii) a common primer region, wherein the first and second circular probes or first and second circularizable probes or probe sets comprise the same primer binding sequence, and wherein the common primer region is capable of hybridizing to the primer binding sequence, wherein the common hybridization region is capable of non-specifically hybridizing to a sequence adjacent to the first and/or second target sequence, (b) photo-activating the photoreactive nucleotide of the first oligonucleotide to react with a nucleotide in the first complementary strand and/or photo-activating the photoreactive nucleotide of the second oligonucleotide to react with a nucleotide in the second complementary strand, thereby crosslinking the first and/or second oligonucleotide to the first and/or second complementary strand, respectively; (c) generating a first and second rolling circle amplification (RCA) product in the biological sample, using the common primer region as a primer and the first or second circular probe or a circularized probe generated from the first or second circularizable probe or probe set as a template, wherein the RCA product comprises the oligonucleotide or a portion thereof crosslinked to the target nucleic acid; (d) contacting the biological sample with a detection probe that hybridizes to the RCA product; and (e) dehybridizing the detection probe from the RCA product while the RCA product remains crosslinked to the target nucleic acid in the biological sample. In some embodiments, the method can further comprise contacting the sample with a subsequent detection probe that hybridizes to the RCA product and dehybridizing the subsequent detection probe from the RCA product while the RCA product remains crosslinked to the target nucleic acid in the biological sample.

In any of the preceding embodiments, the first oligonucleotide and the second oligonucleotide can be the same. In any of the preceding embodiments, the universal base can be pseudouridine. In any of the preceding embodiments, the photoreactive nucleotide can be a modified nucleotide comprising a psoralen or a psoralen derivative. In any of the preceding embodiments, the photoreactive nucleotide can comprise a psoralen or a psoralen derivative connected to a nucleotide via a linker. In any of the preceding embodiments, the photoreactive nucleotide can be a modified nucleotide comprising a vinylcarbazone-based moiety. In any of the preceding embodiments, the vinylcarbazone-based moiety can comprise a 3-cyanovinylcarbazole (^{CNV}K) nucleoside, 3-cyanovinylcarbazole modified D-threoninol (^{CNV}D), a pyranocarbazole nucleoside (^{PC}X) or a pyranocarbazole modified D-threoninol (^{PCX}D).

In some aspects, provided herein is a method for analyzing a biological sample, comprising: (a) contacting the biological sample with: a circular probe or circularizable probe or probe set, and an oligonucleotide comprising (i) a hybridization region that hybridizes to a target nucleic acid in the biological sample and (ii) a primer sequence that hybridizes to the circular probe or circularizable probe or probe set, wherein the hybridization region comprises a photoreactive nucleotide; (b) generating a rolling circle amplification (RCA) product in the biological sample, using the primer sequence as a primer and the circular probe or a circularized probe generated from the circularizable probe or probe set as a template, wherein the RCA product comprises the oligonucleotide or a portion thereof, (c) photo-activating the photoreactive nucleotide to react with a nucleotide in the target nucleic acid, thereby crosslinking the RCA product to the target nucleic acid; (d) contacting the biological sample with a detection probe that hybridizes to the RCA product; and (e) dehybridizing the detection probe from the RCA product while the RCA product remains crosslinked to the target nucleic acid in the biological sample. In some embodiments, the method can further comprise contacting the sample with a subsequent detection probe that hybridizes to the RCA product and dehybridizing the subsequent detection probe from the RCA product while the RCA product remains crosslinked to the target nucleic acid in the biological sample.

In any of the preceding embodiments, the dehybridizing in step (e) can comprise contacting the biological sample with a denaturing agent and/or heating the biological sample. In any of the preceding embodiments, the photoreactive nucleotide may comprise a 3-cyanovinylcarbazole (^{CNV}K) nucleoside, 3-cyanovinylcarbazole modified D-threoninol (^{CNV}D) or a pyranocarbazole (^{PC}X) nucleoside, or a pyranocarbazole modified D-threoninol (^{PCX}D).

In any of the preceding embodiments, the method can further comprise washing the biological sample under a stringent condition prior to the photo-activating and generating the RCA product, wherein the oligonucleotide and/or the circular probe or circularizable probe or probe set that are not stably hybridized to the complementary strand and/or to one another are removed. In any of the preceding embodiments, the method can further comprise ligating the circularizable probe or probe set to generate the circularized probe. In any of the preceding embodiments, the target nucleic acid can be an mRNA crosslinked in the biological sample prior to the photo-activating.

In any of the preceding embodiments, the biological sample may be a fixed and/or permeabilized biological sample. In any of the preceding embodiments, the biological sample may be a tissue sample. In any of the preceding embodiments, the biological sample may be a formalin-fixed, paraffin-embedded (FFPE) tissue sample, a frozen tissue sample, or a fresh tissue sample. In any of the preceding embodiments, the tissue sample may be a tissue slice between about 1 μm and about 50 μm in thickness. In some embodiments, the tissue slice may be between about 5 μm and about 35 μm in thickness. In any of the preceding embodiments, the biological sample may be crosslinked. In any of the preceding embodiments, the biological sample may be embedded in a matrix or not embedded in a matrix. In any of the preceding embodiments, the biological sample may be embedded in a hydrogel. In any of the preceding embodiments, the biological sample may be not embedded in a hydrogel. In any of the preceding embodiments, the biological sample may be cleared. In any of the preceding embodiments, the biological sample may be a hydrogel embedded and cleared sample.

### BRIEF DESCRIPTION OF THE DRAWINGS

The drawings illustrate certain embodiments of the features and advantages of this disclosure. These embodiments are not intended to limit the scope of the appended claims in any manner.

FIG. 1A shows an exemplary nucleic acid concatemer (e.g., rolling circle amplification (RCA) product, optionally a nanoball) dissociating from a complementary strand (e.g., a target mRNA) in a biological sample, wherein the biological sample has been subjected to denaturing conditions (e.g., such as during in situ analysis involving sequential hybridization cycles).

FIG. 1B shows an exemplary nucleic acid concatemer photo-crosslinked to a complementary strand (e.g., a target mRNA) in a biological sample, wherein the photo-crosslinking comprises photo-activating a photoreactive nucleotide in a hybridization region of the nucleic acid concatemer. The crosslinked nucleic acid concatemer remains immobilized on the complementary strand when the biological sample has been subjected to denaturing conditions, thus maintaining the spatial fidelity of the nucleic acid concatemer with respect to the complementary strand.

FIG. 2 shows an exemplary circular or circularized probe and oligonucleotide, wherein the oligonucleotide is crosslinked to the complementary strand.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Provided herein are methods involving the use of a set of polynucleotides comprising at least one modified nucleotide, such as a photoreactive nucleotide, for analyzing one or more target nucleic acid(s) (for example, a messenger RNA or analyte comprising a nucleic acid) present in a sample (e.g., a cell or a biological sample, such as a tissue sample). The methods relate, at least in some aspects, to covalently anchoring a primer used for rolling circle amplification to a complementary strand. In some cases, anchoring a primer allows extension to occur without stalling the reaction which might occur if a nucleic acid used as a template for the extension was anchored. Also provided are polynucleotides, sets of polynucleotides, compositions, kits, systems, and devices for use in accordance with the provided methods. In some aspects, the provided methods can be applied to photo-crosslink a nucleic acid concatemer to a complementary strand during analysis of a target nucleic acid, to maintain the spatial fidelity of the nucleic acid concatemer during downstream analyses (e.g., in situ analysis).

In some embodiments, a biological sample subjected to in situ analysis is not embedded in a polymer matrix such as a hydrogel during sample preparation. For instance, a rolling circle amplification (RCA) product may not be crosslinked to a polymer matrix. During the sequential probe hybridization and removal cycles (e.g., by stripping a set of hybridized probes and hybridization of another set of probes for decoding barcode sequences), the RCA product may be denatured from its circular template and/or a target nucleic acid (e.g., mRNA). While RCA products typically are large molecules and do not easily diffuse, even a modest relocation of an RCA product's position might complicate analysis and possibly compromise the decoding of the RCA product. Thus, shifts in the location of an RCA product in a biological sample during in situ analysis should be minimized.

Although a polymer matrix can be used to embed a sample and immobilize an RCA product to the matrix, this may add significant complexity and time to the tissue preparation and requires both modified surfaces and additional chemistry modifications (e.g., modified nucleotides) to be added during generation of the RCA product. Provided herein in some aspects are compositions and methods for maintaining the association of RCA products in the sample with their respective target nucleic acid molecules (e.g., mRNAs) without the additional complexity of embedding the sample in a matrix such as a hydrogel. In some aspects, provided herein are methods for processing a biological sample, comprising: (a) generating a nucleic acid concatemer (e.g., an RCA product) in the biological sample, wherein the nucleic acid concatemer comprises a photoreactive nucleotide (e.g., ^{CNV}K or ^{CNV}D) in a hybridization region hybridized to a complementary strand; and (b) photo-activating the photoreactive nucleotide to react with a nucleotide in the complementary strand, thereby crosslinking the nucleic acid concatemer to the complementary strand. In some embodiments, the nucleic acid concatemer is an RCA product of a circular or circularized probe or probe set that hybridizes to an mRNA in the biological sample.

### II. Samples, Analytes, and Target Sequences

A method disclosed herein may be used to process and/or analyze any analyte(s) of interest, for example, for detecting the analyte(s) in situ in a sample of interest. A target nucleic acid sequence for an oligonucleotide comprising a photoreactive nucleotide as disclosed herein may be or be comprised in an analyte (e.g., a nucleic acid analyte, such as genomic DNA, mRNA transcript, or cDNA, or a product thereof, e.g., an extension or amplification product) and/or may be or be comprised in a labeling agent for one or more analytes (e.g., a nucleic acid analyte or a non-nucleic acid analyte) in a sample or a product of the labeling agent. Exemplary analytes and labeling agents are described below.

A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular probe or circularizable probe or probe set (e.g., padlock probe). In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circular probe or circularizable probe or probe set (e.g., padlock probe).

In some embodiments, one or more post-fixing step is performed after contacting a sample with a binding or labeling agent (e.g., an antibody or antigen binding fragment thereof) for a non-nucleic acid analyte such as a protein analyte. The labeling agent can comprise a nucleic acid molecule (e.g., reporter oligonucleotide) comprising a sequence corresponding to the labeling agent and therefore corresponds to (e.g., uniquely identifies) the analyte. In some embodiments, the labeling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 m to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but not limited to, acridine orange, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described, e.g., in Chen et al., Science 347(6221):543-548, 2015 and U.S. Pat. No. 10,059,990, which are herein incorporated by reference in their entireties.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016 and U.S. Pat. No. 10,059,990, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probes and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g., PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of species (such as probes) into the sample. In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to opening up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected. In some embodiments, an analyte comprises or is associated with the complementary strand described herein.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

In some aspects, when two or more analytes are analyzed, a first and second probe that is specific for (e.g., specifically hybridizes to) each RNA or cDNA analyte can be used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis (e.g., the reporter sequence may be in the complementary strand hybridized by a nucleic acid concatemer described herein). Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include extension or amplification of templated ligation products to generate the nucleic acid concatemer (e.g., by rolling circle amplification of a circular product generated in a templated ligation reaction).

(i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample. In some embodiments, the DNA analyte comprises the complementary strand described herein.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA). In some embodiments, the RNA analyte comprises the complementary strand described herein.

In some embodiments described herein, an analyte (e.g., an analyte comprising the complementary strand) may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

(ii) Labeling Agents

In some embodiments, provided herein are methods and compositions for analyzing endogenous analytes (e.g., RNA, ssDNA, and cell surface or intracellular proteins and/or metabolites) in a sample using one or more labeling agents. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or epitope-binding fragments thereof. The antibodies or epitope-binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labeling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labeling Agent

In some embodiments, provided herein are methods and compositions for analyzing one or more products of an endogenous analyte and/or a labeling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labeling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed. In some embodiments, a nucleic acid concatemer is generated and anchored in the process of analyzing an endogenous analyte and/or a labelling agent in the sample. In some examples, the complementary strand crosslinked to a nucleic acid concatemer can be an endogenous analyte or a derivative thereof, or a reporter oligonucleotide associated with a labelling agent that indirectly or directly binds to an analyte in the sample.

### III. Nucleic Acid Processing and Analysis

A. Concatemer Generation

In some aspects, provided herein are methods and compositions for processing a biological sample comprising a nucleic acid concatemer, such as a product (e.g., an amplification product such as an RCA product) associated with an analyte in the biological sample. In some aspects, the nucleic acid concatemer is an RCA product of one or more probes associated with (e.g., hybridized to or otherwise bound to) an analyte in the biological sample. The nucleic acid concatemer may be crosslinked to a complementary strand (e.g., in a target nucleic acid) in the biological sample. In some embodiments, the nucleic acid concatemer is a product associated with a labeling agent that directly or indirectly binds to an analyte in the biological sample. In some embodiments, the analyte is a nucleic acid analyte or a non-nucleic acid analyte, and wherein the labeling agent comprises a reporter oligonucleotide and an analyte-binding moiety coupled thereto.

In some embodiments, the nucleic acid concatemer comprises nucleic acids. In some embodiments, the nucleic acid concatemer contains natural and unnatural nucleotides. For example, the nucleic acid concatemer may comprise modified nucleotides, non-nucleotides, or synthetic nucleotides. Nucleotides amendable to the present application include the natural nucleotides of DNA (deoxyribonucleic acid), including adenine (A), guanine (G), cytosine (C), and thymine (T), and the natural nucleotides of RNA (ribonucleic acid), adenine (A), uracil (U), guanine (G), and cytosine (C). Additional suitable bases include natural bases, such as deoxyadenosine, deoxythymidine, deoxyguanosine, deoxycytidine, inosine, diamino purine; base analogs, such as 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 0(6)-methylguanine, 4-((3-(2-(2-(3-aminopropoxy)ethoxy)ethoxy)propyl)amino)pyrimidin-2(1H)-one, 4-amino-5-(hepta-1,5-diyn-1-yl)pyrimidin-2(1H)-one, 6-methyl-3,7-dihydro-2H-pyrrolo[2,3-d]pyrimidin-2-one, 3H-benzo[b]pyrimido[4,5-e][1,4]oxazin-2(10H)-one, and 2-thiocytidine; modified nucleotides, such as 2′-substituted nucleotides, including 2′-O-methylated bases and 2′-fluoro bases; and modified sugars, such as 2′-fluororibose, ribose, 2′-deoxyribose, arabinose, and hexose; and/or modified phosphate groups, such as phosphorothioates and 5′-N-phosphoramidite linkages. In some embodiments, the modified nucleotides are amine-modified nucleotides.

In some embodiments, the nucleic acid concatemer is between about 1 and about 85 kilobases in length, such as between any of about 1 and about 15 kilobases, about 10 and about 30 kilobases, about 20 and about 40 kilobases, about 30 and about 50 kilobases, about 40 and about 60 kilobases, about 50 and about 70 kilobases, and about 60 and about 85 kilobases in length. In some embodiments, the nucleic acid concatemer is at least 1 kilobase in length, such as any of about 15, 25, 35, 45, 55, 65, or 85 kilobases in length. In some embodiments, the nucleic acid concatemer is more than 85 kilobases in length.

In some embodiments, the nucleic acid concatemer is a rolling circle amplification product comprising a barcode sequence corresponding to an analyte in the biological sample, and the nucleic acid concatemer is between about 1 and about 15 kilobases, between about 15 and about 25 kilobases, between about 25 and about 35 kilobases, between about 35 and about 45 kilobases, between about 45 and about 55 kilobases, between about 55 and about 65 kilobases, between about 65 and about 75 kilobases, or more than 75 kilobases in length.

In some embodiments, the nucleic acid concatemer forms a nanoball such as one having a diameter between about 0.1 μm and about 3 μm. The nanoball may be a product of amplification, such as RCA. In some embodiments, the nanoball has a diameter of between about 0.1 μm and about 4 μm, such as between any of about 0.1 μm and about 0.5 μm, about 0.2 μm and about 2 μm, about 1 μm and about 3 μm, and about 2 μm and about 4 μm. In some embodiments, the nanoball diameter is at least about 0.1 μm, such as at least any of about 0.2 μm, 0.5 μm, 1 μm, 1.5 μm, 2 μm, 2.5 μm, 3 μm, 3.5 μm, and 4 μm.

In some embodiments, as described in Section II.B, the analyte associated with the nucleic acid concatemer may be a nucleic acid analyte. For example, the nucleic acid analyte may be DNA, ssDNA, or RNA. In some embodiments, the analyte is a non-nucleic acid analyte (e.g., a protein). In some embodiments, the nucleic acid concatemer is generated from a labeling agent that directly or indirectly binds to an analyte in the biological sample. Examples of labeling agents are described in Section II.B.(ii).

In some aspects, the polynucleotides and/or amplification product (e.g., amplicon) can be further anchored to a polymer matrix. For example, the polymer matrix can be a hydrogel. In some embodiments, a nucleic acid molecule (e.g., the complementary strand to which the concatemer is crosslinked to) in the sample can be modified to contain functional groups (e.g., photoreactive nucleotides) that can be used as an anchoring site to attach to a polymer matrix. Exemplary polymer matrix that can be employed in accordance with the provided embodiments comprise those described in, for example, US 2016/0024555, US 2018/0251833, US 2016/0024555, US 2018/0251833 and US 2017/0219465, all of which are herein incorporated by reference in their entireties. In some examples, the scaffold can comprise oligonucleotides, polymers or chemical groups, to provide a matrix and/or support structures. In some embodiments, the biological sample is not embedded in a polymer matrix.

In some preferred embodiments, the nucleic acid concatemer is an amplification product, such as a rolling circle amplification (RCA) product. RCA may comprise contacting the biological sample with one or more probes to produce an RCA product (RCP). In some embodiments, the methods disclosed herein involve the use of one or more probes or probe sets that directly or indirectly hybridize to a target nucleic acid, such as a nucleic acid analyte. The probes or probes sets can be used to generate a circularized probe for rolling circle amplification, thereby generating the nucleic acid concatemer. Various probes and probe sets can be hybridized to a complementary strand (e.g., an endogenous analyte and/or a labeling agent) and each probe may comprise one or more barcode sequences. Exemplary barcoded probes or probe sets may be modified probe sets based on a padlock probe, a gapped padlock probe, a SNAIL (Splint Nucleotide Assisted Intramolecular Ligation) probe set, a PLAYR (Proximity Ligation Assay for RNA) probe set, and a PLISH (Proximity Ligation in situ Hybridization) probe set. The specific probe or probe set design can vary. In some aspects, the biological sample is contacted with a barcoded probe or barcoded probe set. In some embodiments, the barcode probe or barcoded probe set comprises one or more barcode sequences. Therefore, in some embodiments, the nucleic acid concatemer comprises one or more barcode sequences. The barcode sequence may be specific to a target nucleic acid (e.g., an analyte) in the biological sample, thereby serving to identify the target nucleic acid during downstream analyses.

In some embodiments, the one or more probes or probe sets comprise a circular probe, or a circularizable probe or probe set. In some embodiments, the one or more probes or probe sets (e.g., comprising the circular probe or the circularizable probe or probe set) comprises an oligonucleotide. In some embodiments, a circularizable probe or probe set may be ligated to form a circularized probe. For example, various probe or probe sets are capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is based on a SNAIL probe set, for example, wherein the oligonucleotide comprises a primer region or is linked (e.g., ligated) to a primer that templates ligation of a circularizable probe. In some cases, SNAIL probe designs, see, e.g., U.S. Pat. Pub. 20190055594, which is hereby incorporated by reference in its entirety. In some embodiments, provided herein is a probe or probe set capable of proximity ligation, for instance a probe set based on a proximity ligation assay for RNA (e.g., PLAYR) probe set. See, e.g., U.S. Pat. Pub. 20160108458, which is hereby incorporated by reference in its entirety. In some embodiments, the oligonucleotide can comprise a primer region of a PLAYR probe set or be linked (e.g., ligated) to a primer of a PLAYR probe set. In some embodiments, a circular probe can be indirectly hybridized to the target nucleic acid. In some embodiments, the circular construct can be formed from a probe set capable of proximity ligation, for instance a probe set based on a proximity ligation in situ hybridization (PLISH) probe set described, for example, in U.S. Pat. Pub. 2020/0224243 which is hereby incorporated by reference in its entirety. For example, the oligonucleotide can comprise a primer region of an H-probe pair in a PLISH probe set, or can be linked (e.g., ligated) to a primer region of an H-probe pair.

In some embodiments, the ligation is performed prior to photo-activating the photoreactive nucleotide to react with a nucleotide in the complementary strand. In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase can comprise an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. Direct ligation can comprise ligating the ends of polynucleotides hybridized immediately adjacently to one another to form a substrate for a ligase enzyme resulting in their ligation to each other (intramolecular ligation). Indirect ligating the ends of the polynucleotides that hybridize non-adjacently to one another, e.g., separated by one or more intervening nucleotides or gaps. In some embodiments, said ends are not ligated directly to each other, but instead are ligated either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be filled by one or more gap (oligo)nucleotide(s) which are complementary to a splint, a circularizable probe or probe set (e.g., padlock probe), or a target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the circular probe or the circularizable probe or probe set comprises a barcode sequence. In some embodiments, the barcode sequence of the circular probe or the circularizable probe or probe set is incorporated into the nucleic acid concatemer during amplification (e.g., RCA). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides. In some cases, a barcode can be or can include a unique molecular identifier or “UMI”. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the preceding embodiments, barcodes (e.g., primary and/or secondary barcode sequences) can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of labelled probes (e.g., detection oligos). In some cases, since the barcode sequences are pre-determined, they can also be designed to feature error detection and correction mechanisms, see, e.g., U.S. Pat. Pub. 20190055594 and U.S. Pat. Pub. 2021016403, which are hereby incorporated by reference in their entirety.

In some embodiments, the one or more probes or probe sets further comprise an oligonucleotide (e.g., an oligonucleotide probe). In some embodiments, the oligonucleotide comprises a hybridization region comprising one or more photoreactive nucleotides, and hybridizes to the complementary strand. In some embodiments, the oligonucleotide comprises a primer region (also referred to herein as a primer sequence) or is linked (e.g., ligated) to a primer. Primers or primer regions can vary in length. For example, primers or primer regions can be about 6 bases to about 120 bases. For example, primers or primer regions can include up to about 25 bases. A primer, may in some cases, refer to a primer binding sequence or primer region. A primer extension reaction generally refers to any method where two nucleic acid sequences become linked (e.g., hybridized) by an overlap of their respective terminal complementary nucleic acid sequences (for example, 3′ termini). Such linking can be followed by nucleic acid extension (e.g., an enzymatic extension) of one, or both termini using the other nucleic acid sequence as a template for extension.

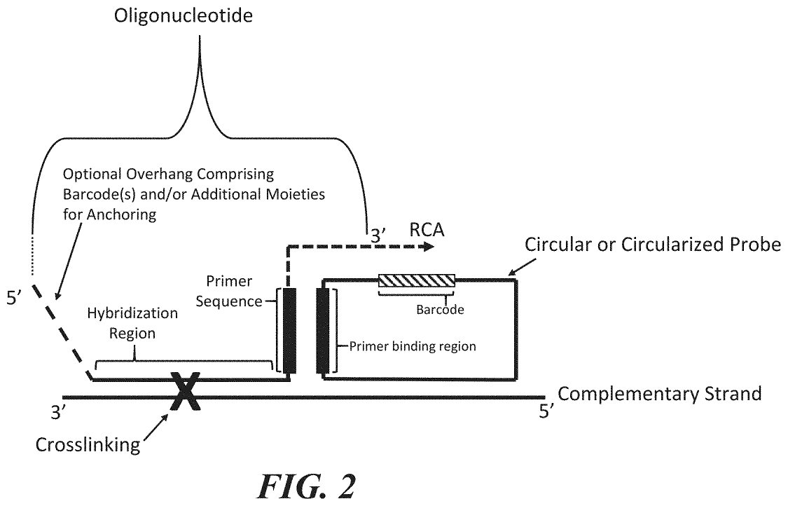

In some embodiments, the oligonucleotide may comprise (or may be ligated to) a primer sequence (also referred to herein as a primer region) for rolling circle amplification (RCA) of the circular or circularized probe that hybridizes to the complementary strand, wherein the primer sequence is complementary to a region of the circular or circularized probe. For example, as shown in FIG. 2, the oligonucleotide can comprise a primer sequence that hybridizes to the circular or circularized probe for rolling circle amplification. The oligonucleotide may further comprise a 5′ end sequence that does not hybridize to the complementary strand that may be used for additional probe hybridization and/or anchoring to a matrix and/or other molecules in the biological sample (an overhang region, as shown in FIG. 2). The circular or circularized probe may comprise one or more barcode sequences that can be used to identify the complementary strand during downstream analyses. The complementary strand may be part of a target nucleic acid molecule (e.g., mRNA) or a labeling agent for a nucleic acid analyte or a non-nucleic acid analyte. In some embodiments, the oligonucleotide comprises a hybridization region that hybridizes to a target nucleic acid in the biological sample and a primer sequence that hybridizes to the circular probe or circularizable probe or probe set. For example, the oligonucleotide comprises from 5′ to 3′: a hybridization region that hybridizes to a target nucleic acid in the biological sample and a primer sequence that hybridizes to the circular probe or circularizable probe or probe set. FIG. 2 shows an exemplary oligonucleotide comprising a hybridization region and a primer sequence. The hybridization region of the oligonucleotide hybridizes to a target nucleic acid (e.g., a complementary strand of a target nucleic acid) in a biological sample. The primer sequence of the oligonucleotide hybridizes to the circular probe, which allows for productive RCA. In some embodiments, non-hybridized oligonucleotide probes are removed from the sample prior to photo-activating the photoreactive nucleotide to crosslink the hybridization region of the oligonucleotide to the complementary strand.

In some embodiments, the oligonucleotide comprising a primer sequence has a 3′ end that can be used as a substrate for a nucleic acid polymerase in a nucleic acid extension reaction. In some embodiments, the primer sequence is located at the 3′ terminal of the oligonucleotide probe. In some aspects, the oligonucleotide probe comprises a hybridization region located 5′ relative to the primer sequence in the oligonucleotide probe. In some cases, any photoreactive nucleotides in the oligonucleotide probe for crosslinking to the complementary strand is located 5′ relative to the primer sequence. In some embodiments, the photoreactive nucleotides comprise a psoralen or psoralen derivative. The photoreactive nucleotides may also comprise of vinylcarbazone-based moieties, such as a cyanovinylcarbazole (^{CNV}K) nucleoside, a cyanovinylcarbazole modified D-threoninol (^{CNV}D), a pyranocarbazole (^{PC}X) nucleoside, or a pyranocarbazole modified D-threoninol (^{PCX}D)

In some embodiments, the hybridization region of the oligonucleotide comprises one or more universal bases. In some embodiments, the hybridization region of the oligonucleotide is composed of universal bases. In some aspects, the hybridization region can hybridize to a nucleic acid sequence in the complementary strand non-specifically (e.g., without sequence-specificity), as the universal bases are able to form base pairs with any of the bases present in the complementary strand (e.g., the universal base can pair with an A, T, G, C, or U). The oligonucleotide can thus comprise a universal hybridization region comprising universal bases, and a priming region, which can be a common priming region. In some embodiments, the common priming region is complementary to a primer binding region present in a plurality of different circular or circularizable probes or probe sets. Thus, a common oligonucleotide can be used to crosslink to the complementary strand and to prime amplification of the circular or circularized probe for a plurality of different complementary strands and corresponding circular or circularizable probes or probe sets. In some embodiments, the universal base or universal bases can be connected to one or more photocrosslinkable moieties (optionally via a linker).

Exemplary universal bases have been described, such as deoxyinosine (Ohtsuka, E. et al., (1985) J. Biol. Chem. 260, 2605-2608; and Sakanari, S. A. et al., (1989) Proc. Natl. Acad. Sci. 86, 4863-4867), 1-(2′-deoxy-beta-D-ribofuranosyl)-3-nitropyrrole (Nichols, R. et al., (1994) Nature 369, 492-493) and 5-nitroindole (Loakes, D. et al., (1994) Nucleic Acids Res. 22, 4039-4043). In some embodiments, the one or more universal bases of the competing region is can comprise deoxyinosine, inosine, 7-deaza-2′-deoxyinosine, 2-aza-2′-deoxyinosine, 2′-OMe inosine, 2′-F inosine, deoxy 3-nitropyrrole, 3-nitropyrrole, 2′-OMe 3-nitropyrrole, 2′-F 3-nitropyrrole, 1-(2′-deoxy-beta-D-ribofuranosyl)-3-nitropyrrole, deoxy 5-nitroindole, 5-nitroindole, 2′-OMe 5-nitroindole, 2′-F 5-nitroindole, deoxy 4-nitrobenzimidazole, 4-nitrobenzimidazole, deoxy 4-aminobenzimidazole, 4-aminobenzimidazole, deoxy nebularine, 2′-F nebularine, 2′-F 4-nitrobenzimidazole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2′-0-methoxyethyl inosine, 2′O-methoxyethyl nebularine, 2′-0-methoxyethyl 5-nitroindole, 2′-0-methoxyethyl 4-nitro-benzimidazole, 2′-0-methoxyethyl 3-nitropyrrole, and combinations thereof. In some embodiments, the one or more universal bases of the competing region can comprise deoxyinosine, 1-(2′-deoxy-beta-D-ribofuranosyl)-3-nitropyrrole or 5-nitroindole. In some embodiments, the one or more universal bases of the competing region can comprise deoxyinosine. In some cases, the universal bases can be pseudouridines or inosines. In some cases, the universal bases are pseudouridines. The universal bases can be connected to psoralens or other sequence independent crosslinkable moieties via a linker. Other sequence independent crosslinkable moieties can include vinylcarbazone-based moieties. Vinylcarbazone-based moieties can include a cyanovinylcarbazole (^{CNV}K) nucleoside, a cyanovinylcarbazole modified D-threoninol (^{CNV}D), or a pyranocarbazole (^{PC}X) nucleoside, or a pyranocarbazole modified D-threoninol (^{PCX}D).

In some aspects, provided herein is a method for analyzing a biological sample, comprising contacting the biological sample with a plurality of circular or circularizable probes or probe sets and a common oligonucleotide, wherein the circular or circularizable probes or probe sets hybridize to specific target sequences in different complementary strands. The common oligonucleotide can comprise a universal hybridization region comprising universal bases (e.g., pseudouridines) and one or more photoreactive moieties (e.g., photocrosslinkable moieties such as psoralens, coumarins, vinylcarbazones, or derivatives thereof). The common oligonucleotide can also comprise a common primer region complementary to a primer binding region that is common among the circular or circularizable probes. In some embodiments, the primer binding region is positioned adjacent to the region of the circular or circularizable probe or probe set that hybridizes to the complementary strand (e.g., 5′ of the region of the circular or circularizable probe or probe set that hybridizes to the complementary strand). The common oligonucleotide can hybridize to the primer binding region in the different circular or circularizable probes or probe sets, and the universal hybridization region can hybridize to the region of the complementary strand that is 3′ of and adjacent to any of the target sequences (e.g., without sequence specificity). Thus, the same common oligonucleotide can be used to crosslink a plurality of different nucleic acid concatemers to the complementary strand. In some aspects, this avoids the need to synthesize different oligonucleotides comprising one or more photoreactive nucleotides for different complementary strands. The remaining steps of photo-activating the photoreactive nucleotide to react with a nucleotide in the complementary strand, generating the rolling circle amplification products (nucleic acid concatemers) for the plurality of circular or circularized probe templates, and detecting the rolling circle amplification products can be carried out as described herein. In some embodiments, the photoreactive nucleotide is a modified pseudouridine comprising a psoralen or psoralen derivative, optionally connected to the pseudouridine via a linker.

In some embodiments, a product of an endogenous analyte and/or a labeling agent is an amplification product of one or more polynucleotides, for instance, a circular probe or circularizable probe or probe set. In some embodiments, the amplifying is achieved by performing rolling circle amplification (RCA) to generate the concatemer. In other embodiments, a primer that hybridizes to the circular probe or circularized probe is added and used as such for amplification. In some embodiments, the RCA comprises a linear RCA, a branched RCA, a dendritic RCA, or any combination thereof. In some aspects, during the amplification step, modified nucleotides can be added to the reaction to incorporate the modified nucleotides in the amplification product for additional anchoring of the concatemer.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) are known in the art such as linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some embodiments, the RCA template may comprise the target analyte, or a part thereof, where the target analyte is a nucleic acid, or it may be provided or generated as a proxy, or a marker, for the analyte. As noted above, many assays are known for the detection of numerous different analytes, which use a RCA-based detection system, e.g., where the signal is provided by generating a RCP from a circular RCA template which is provided or generated in the assay, and the RCP is detected to detect the analyte. The RCP may thus be regarded as a reporter which is detected to detect the target analyte. However, the RCA template may also be regarded as a reporter for the target analyte; the RCP is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

B. Hybridization Region

In some embodiments, the hybridization region of the oligonucleotide, and the nucleic acid concatemer (e.g., RCP) resulting therefrom, hybridizes to a target nucleic acid in a biological sample. In some embodiments, the hybridization region hybridizes to a complementary strand of the target nucleic acid. The complementary strand may comprise the target nucleic acid analyte. In some embodiments, the complementary strand is in a nucleic acid in the biological sample. In some embodiments, the complementary strand is in a single stranded nucleic acid. In some embodiments, the complementary strand is in a double stranded nucleic acid. In some embodiments, the double stranded nucleic acid has been unwound to produce a single stranded complementary strand to which the oligonucleotide probe may hybridize. In some embodiments, the complementary strand is in a cellular RNA, in an mRNA, in a cDNA, or in a genomic DNA in the biological sample. In some embodiments, the complementary strand is in a nucleic acid reporter oligonucleotide comprised by a labeling agent as described in Section II.B.(ii). In some embodiments, the complementary strand does not comprise modified nucleic acids (e.g., photoreactive nucleic acids). In some embodiments, the complementary strand is coupled to a matrix. In some embodiments, the matrix is embedded in the biological sample. In some embodiments, the complementary strand is crosslinked in the biological sample. For example, the complementary strand (e.g., mRNA) can be fixed prior to generating a nucleic acid concatemer. In some embodiments, the crosslinking comprises applying formaldehyde to the biological sample. In some embodiments, the crosslinking does not comprise photo-crosslinking.

The hybridization region of the oligonucleotide hybridizes to the complementary strand. In some embodiments, the hybridization region comprises a nucleic acid sequence. In some embodiments, the nucleic acid sequence comprises natural nucleic acids and unnatural nucleic acids (e.g., photoreactive nucleotides). In some embodiments, the hybridization region comprises photoreactive nucleotides, as described in Section IV. In some embodiments, the hybridization region is a specific hybridization region that hybridizes to a complementary sequence in the complementary strand. In some embodiments, the hybridization region is a universal hybridization region capable of hybridizing to the complementary strand without regard to sequence complementarity according to Watson-Crick base pairing rules. In some embodiments, the hybridization region comprises universal bases. Exemplary universal bases are described above, including inosine and pseudouridine. In some embodiments, the photoreactive nucleotides in the hybridization region comprise universal bases. In some embodiments, the hybridization region comprises one or more pseudouridines modified with a psoralen or psoralen derivative. In some embodiments, the amplification of the target nucleic acid generates the nucleic acid concatemer comprising the hybridization region.

In any of the embodiments herein, the hybridization region may comprise a 5′ end sequence, an internal sequence, and/or a 3′ end sequence of the nucleic acid concatemer. In any of the embodiments herein, the hybridization region may be 5′ to the tandem unit sequences of the nucleic acid concatemer. In any of the embodiments herein, the hybridization region may be at the 5′ end of the nucleic acid concatemer, e.g., as shown in FIG. 2. In some embodiments, the hybridization region may be in an anchor that hybridizes to the complementary strand. In some embodiments, the hybridization region may be in an anchor that hybridizes to a nucleic acid coupled to a matrix embedding the biological sample. In some embodiments, the anchor further comprises a primer region that hybridizes to a circular or circularized probe to prime a rolling circle amplification reaction to generate the nucleic acid concatemer. In some embodiments, the anchor (which may or may not hybridize to a circular or circularized probe) is ligated to a primer that hybridizes to the circular or circularized probe to prime a rolling circle amplification reaction to generate the nucleic acid concatemer. In any of the embodiments herein, the anchor may further comprise a 5′ overhang sequence that does not hybridize to the complementary strand. In any of the embodiments herein, a hybridization region comprising one or more of the photoreactive nucleotide may be provided at the 3′ of the tandem unit sequences of the nucleic acid concatemer, where the hybridization region may hybridize to the complementary strand (e.g., mRNA) and/or the circular or circularized probe (e.g., a circularized padlock probe) and may be crosslinked thereto via photo-activating the photoreactive nucleotide(s).

In some embodiments, the hybridization region is between about 5 and about 30 nucleotides in length, such as between any of about 5 and about 20, about 10 and about 25, and about 15 and about 30 nucleotides in length. In some embodiments, the hybridization region is between about 10 and about 15 nucleotides in length. In some embodiments, the hybridization region is at least about 5 nucleotides in length, such as at least any of about 10, 15, 20, 25, or 30 nucleotides in length.

In any of the embodiments herein, the hybridization region may comprise two or more photoreactive nucleotides. In any of the embodiments herein, the hybridization region may comprise three, four, five or more photoreactive nucleotides. In any of the embodiments herein, the hybridization region may comprise about 2, 5, 10, 15, 20, 25, or 30 photoreactive nucleotides, or any range in between. Any one or more of the photoreactive nucleotides can be ^{CNV}D, ^{CNV}K, or any other suitable photoreactive nucleotides.

### IV. Photoreactive Nucleotides and Photo-Activated Crosslinking

The hybridization region of the nucleic acid concatemer may further comprise one or more photoreactive nucleotides. In some aspects, the methods provided herein comprise crosslinking a nucleic acid concatemer to a biological sample. In some embodiments, the crosslinking occurs in the hybridization region of the nucleic acid concatemer. In some embodiments, the nucleic acid concatemer is crosslinked upon activation by providing a stimulus. In some embodiments, the nucleic acid concatemer is crosslinked to the biological sample via the one or more photoreactive nucleotides in the hybridization region. The photoreactive nucleotide(s) may become photo-activated as described in Section IV, in order to crosslink the nucleic acid concatemer to the target nucleic acid in the biological sample. In some embodiments, the photoreactive nucleotide crosslinks the nucleic acid concatemer to the biological sample, a substrate, and/or a matrix.

In some embodiments, activation of the photoreactive nucleotide is light driven and can be performed in aqueous solution. In some embodiments, crosslinking strands of nucleic acid molecules comprise at least one photo-reactive nucleobase. In some embodiments, the photo reactive nucleobase can be any modified nucleobase that is capable of forming a crosslink with another nucleobase in the presence of light. In some embodiments, the photo-reactive nucleobase can be a modified pyrimidine or purine nucleobase. In some embodiments, the photo reactive nucleobase can comprise a vinyl, acrylate, N-hydroxysuccinimide, amine, carboxylate or thiol chemical group. In some embodiments, the photo-reactive nucleobase comprises a bromo-deoxyuridine.

In some embodiments, the photoreactive nucleotide comprises a reactive chemical group that requires light activation to initiate crosslinking. In some embodiments, the chemical group comprises, for example, an aryl azide, azido-methyl-coumarin, benzophenone, anthraquinone, certain diazo compounds, diazirine, or a psoralen derivative.

In some embodiments, the photoreactive nucleotide comprises a cyanovinylcarbazole moiety. In some embodiments, the photoreactive nucleotide comprises a 3-cyanovinylcarbazole (^{CNV}K) nucleoside, 3-cyanovinylcarbazole modified D-threoninol (^{CNV}D), a pyranocarbazole (^{PC}X) nucleoside, or a pyranocarbazole modified D-threoninol (^{PCX}D). In some embodiments, the photoreactive nucleotide comprises 3-cyanovinylcarbazole phosphoramidite. In some embodiments, the photoreactive nucleotide comprises pyranocarbazole phosphoramidite. In some embodiments, the photoreactive nucleotide may comprise a 3-cyanovinylcarbazole (^{CNV}K) nucleoside, a 3-cyanovinylcarbazole modified D-threoninol (^{CNV}D), or a 3-cyanovinylcarbazole phosphoramidite that may be light activated. In some embodiments, the photoreactive nucleotide may comprise a pyranocarbazole (^{PC}X) nucleoside, a pyranocarbazole modified D-threoninol (^{PCX}D), or a pyranocarbazole phosphoramidite and may be light activated. In some embodiments, the photoreactive nucleotides have been attached (e.g., by extension with a polymerase or ligation) to a probe (e.g., an oligonucleotide probe) that is hybridized to a target nucleic acid within a sample. In some embodiments, the photoreactive nucleotides have been attached to the oligonucleotide via a linker (e.g., a disulfide linker).

In some embodiments, activation of the photoreactive nucleotide is light driven and can be performed in aqueous solution. In some embodiments, crosslinking strands of nucleic acid molecules comprise at least one photo-reactive nucleobase. In some embodiments, the photo reactive nucleobase can be any modified nucleobase that is capable of forming a crosslink with another nucleobase in the presence of light. In some embodiments, the photo-reactive nucleobase can be a modified pyrimidine or purine nucleobase. In some embodiments, the photo reactive nucleobase can comprise a vinyl, acrylate, N-hydroxysuccinimide, amine, carboxylate or thiol chemical group. In some embodiments, the photo-reactive nucleobase comprises a bromo-deoxyuridine. Exemplary photoreactive moieties and photoreactive nucleotides are described, for example, in Elskens and Madder RSC Chem. Biol., 2021, 2, 410-422, the content of which is herein incorporated by reference in its entirety.

In some embodiments, the photoreactive nucleotide comprises a reactive chemical group that requires light activation to initiate crosslinking. In some embodiments, the chemical group comprises, for example, an aryl azide, azido-methyl-coumarin, benzophenone, anthraquinone, certain diazo compounds, diazirine, or a psoralen derivative. In some embodiments, the photoreactive nucleotide comprises a cyanovinylcarbazole moiety. In some embodiments, the photoreactive nucleotide comprises a 3-cyanovinylcarbazole (^{CNV}K) nucleoside or 3-cyanovinylcarbazole modified D-threoninol (^{CNV}D). In some embodiments, the photoreactive nucleotide comprises 3-cyanovinylcarbazole phosphoramidite. In some embodiments, the photoreactive nucleotide comprises a pyranocarbazole. In some embodiments, the photoreactive nucleotide comprises a pyranocarbazole (^{PC}X) modified nucleoside or a pyranocarbazole with a D-threoninol instead of a 2′-deoxyribose backbone (^{PCX}D). In some embodiments, the photoreactive nucleotide comprises a psoralen or a coumarin. In some embodiments, the photoreactive nucleotides have been attached (e.g., by extension with a polymerase or ligation) to a probe (e.g., an oligonucleotide probe) that is hybridized to a target nucleic acid within a sample. In some embodiments, the photoreactive nucleotide comprises a universal base. In some embodiments, an oligonucleotide comprising one or more photoreactive nucleotides comprising universal bases is able to hybridize to a plurality of different sequences in a complementary strand (e.g., by hybridization of the universal base to the sequence in the complementary strand), wherein the complementary strand is complementary to a circular probe or circularizable probe or probe set. The oligonucleotide can comprise a common primer region capable of hybridizing to a primer binding region present in a plurality of the circular probes or circularizable probes or probe sets. In some embodiments, the oligonucleotide can be used as a common primer for rolling circle amplification and crosslinking of a plurality of circular or circularized probes for a plurality of different complementary strands (e.g., different target RNAs).

The ^{PC}X crosslinking base displays high crosslinking efficiency with a thymine (T) base or a cytosine (C) base that is positioned adjacent to the base on the complementary strand and can be directly incorporated into the DNA hybridization domain itself as a base substitution. In some embodiments, a crosslinking reaction is performed using 400 nm wavelength of light and can be completed within about 10 seconds. In some embodiments, a crosslinking reaction can be completed within 0.1, 0.25, 0.5, 1, 5, or 10 seconds. In some embodiments, a crosslinking reaction can be completed within 0.5, 1, 5, 10, 20, 30, 40, 50, or 60 minutes. In some embodiments, a crosslinking reaction can be completed within 0.5, 1, 2, 3, 4, or 5 minutes. In some embodiments, a crosslinking reaction has negligible effects on bases that neighbor the photoreactive nucleobase. In some embodiments, other photochemical nucleic acid crosslinking agents, including psoralen can be used in combination with the photoreactive nucleobases disclosed herein. In some embodiments, a photo-induced crosslink can be reversed. In some embodiments, a ^{PC}X crosslink can be reversed when exposed to 312305 nm UV light.

In some embodiments, other photochemical nucleic acid crosslinking agents, including psoralen and psoralen derivatives (e.g., psoralen modified nucleosides) can be used in combination with the photoreactive nucleobases disclosed herein, Psoralen and psoralen derivatives can be light-activated with a UV-A of 365 nm. Psoralens react with nearby pyrimidine residues. A variety of nucleosides modified with psoralen or psoralen derivatives may be used. For example, click chemistry using a psoralen azide and a nucleosidic alkyne derivative can be used to generate a variety of photoreactive nucleotides. The psoralen can be connected to the nucleotide via a linker, such as a phosphoramidite. Exemplary psoralen derivatives comprising phosphoramidite include but are not limited to 6-[4′-(Hydroxymethyl)-4,5′,8-trimethylpsoralen]-hexyl-1-O-(2-cyanoethyl)-(N,N-diisopropyl)-phosphoramidite and 2-[4′-(hydroxymethyl)-4,5′,8-trimethylpsoralen]-ethyl-1-O-(2-cyanoethyl)-(N,N-diisopropyl)-phosphoramidite. In some embodiments, the psoralen or psoralen derivative is conjugated to position 5 of a uridine or pseudouridine (optionally via a linker). In some cases, the psoralen or psoralen derivative is conjugated to the 2′ position of a sugar ring of a uridine or pseudouridine (optionally via a linker). In some embodiments, the psoralen derivative can be an amine-reactive derivative, which can be conjugated to an amine-modified nucleotide (e.g., an aminoallyl uridine or pseudouridine nucleotide).

In some embodiments, a psoralen-crosslink can be reversed when exposed to 254 nm light. In some embodiments, the photoactivatable nucleotide comprises a C2′ psoralen modification. The photoactivatable nucleotide can comprise a 5′ psoralen derivative, and can be at the 5′ end of the hybridization region. The structure of two exemplary psoralen-modified oligonucleotides (one 5′ modified nucleoside on the left, and one C2′ modified nucleoside on the right) are shown below:

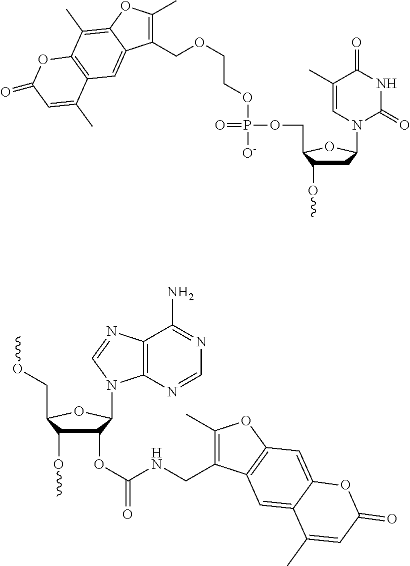

In some embodiments, the photoactivatable nucleotide is a universal base such as a pseudouridine modified with a photoreactive moiety (e.g. a psoralen).

In some embodiments, when ^{CNV}K is incorporated into an oligonucleotide, rapid photo cross-linking to pyrimidines in the complementary strand (DNA or RNA) can be induced at one wavelength and rapid reversal of the cross-link is possible at a second wavelength if desired. Neither wavelength has the potential to cause significant DNA damage and neither interfere with the wavelengths used to excite the fluorophores used during subsequent analysis, such as decoding barcode sequences in situ. Once cross-linked, the UV melting temperature of the duplex may be raised by around 30° C./^{CNV}K moiety relative to the duplex before irradiation and inter-strand crosslinking. The structure of an exemplary 3-cyanovinylcarbazole phosphoramidite is shown below:

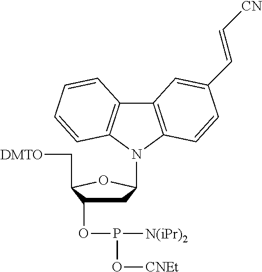

### 5′-O-(4,4′-Dimethoxytrityl)-1′-(3-cyanovinylcarbazol-9-yl)-2′-deoxy-β-D-ribofuranosyl-3′-[(2-cyanoethyl)-(N,N-diisopropyl)]-phosphoramidite

The ^{CNV}K crosslinking base displays high crosslinking efficiency with a thymine (T) base that is positioned adjacent to the base on the complementary strand (Ultrafast reversible photo-cross-linking reaction: toward in situ DNA manipulation. Org. Lett. 10, 3227-3230 (2008)) and can be directly incorporated into the DNA hybridization domain itself as a base substitution, as shown below in light-directed reaction between a ^{CNV}K base modification and a thymine base to produce a crosslinked nucleic acid.

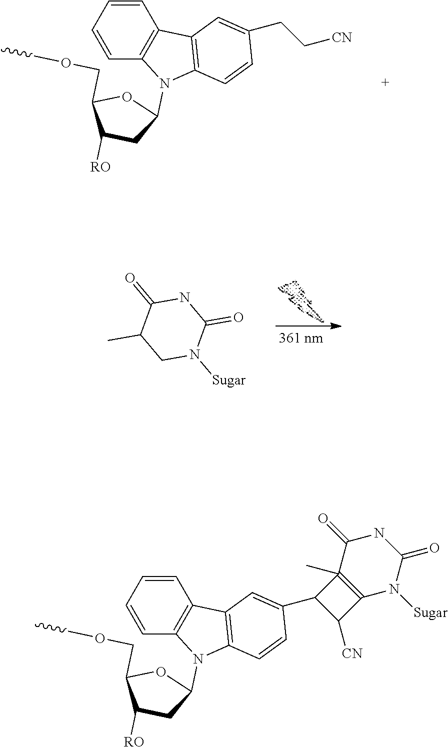

In some embodiments, a crosslinking reaction is performed using 365 nm wavelength of light and can be completed within about 1 second. In some embodiments, a crosslinking reaction can be performed using any wavelength of visible or ultraviolet light. In some embodiments, a crosslinking reaction can be completed within 0.1, 0.25, 0.5, 1, 5, or 10 seconds. In some embodiments, a crosslinking reaction can be completed within 20, 30, 40, 50, or 60 seconds. In some embodiments, the method comprises irradiating the biological sample with UV light, such as a 350-400 nm wavelength of light, for between 10 seconds and 10 minutes, between 10 seconds and 5 minutes, between 10 seconds and 2 minutes, between 10 seconds and 1 minute, between 30 seconds and 1 minute, or between 30 seconds and 5 minutes. In some embodiments, a crosslinking reaction can be completed within 0.5, 1, 5, 10, 20, 30, 40, 50, or 60 minutes. In some embodiments, a crosslinking reaction has negligible effects on bases that neighbor the photoreactive nucleobase. In some embodiments, other photochemical nucleic acid crosslinking agents, including psoralen and coumarin can be used in combination with the photoreactive nucleobases disclosed herein.

In some embodiments, the photoreactive nucleotide comprises a coumarin and the photoactivation comprises irradiating the biological sample using a 350 nm wavelength of light. In some embodiments, the photoreactive nucleotide comprises a psoralen and the photoactivation comprises irradiating the biological sample using a 365 nm wavelength of light. In some embodiments, the photoreactive nucleotide comprises a ^{CNV}K or ^{CNV}D and the photoactivation comprises irradiating the biological sample using a 365 nm wavelength of light. In some embodiments, the photoreactive nucleotide comprises a ^{PC}X or ^{PCX}D and the photoactivation comprises irradiating the biological sample using a 400 nm wavelength of light. In some embodiments, the photoreactive nucleotide comprises a diazirine and the photoactivation comprises irradiating the biological sample using a 365 nm wavelength of light. In some embodiments, the photoreactive nucleotide comprises a thiouridine and the photoactivation comprises irradiating the biological sample using a 365 nm wavelength of light.

In some embodiments, a photo-induced crosslink can be reversed. In some embodiments, a vinylcarbazole (e.g., ^{CNV}K ^{CNV}D, ^{PC}X, or ^{PCX}D) crosslink can be reversed when exposed to 305 nm UV light. In some embodiments, a vinylcarbazole (e.g., ^{CNV}K, ^{CNV}D, ^{PC}X, or ^{PCX}D) crosslink can be reversed when exposed to 312 nm light. In some embodiments, a psoralen crosslink can be reversed when exposed to 254 nm light. In some embodiments, a coumarin crosslink can be reversed when exposed to 254 nm light.

In some embodiments, the photoactivatable nucleotide comprises a coumarin and hybridizes to a thymine (T) base in the complementary strand. In some embodiments, the photoactivatable nucleotide comprises a psoralen and hybridizes to a C, T, or U base in the complementary strand. In some embodiments, the photoactivatable nucleotide comprises a vinylcarbanazole and hybridizes to a C, T, or U base in the complementary strand.

In some embodiments, the nucleic acid concatemer comprises at least one photoreactive nucleotide. In some embodiments, the at least one photoreactive nucleotide is present in the hybridization region (e.g., in the 5′ end sequence, in the internal sequence, and/or a 3′ end sequence) of the nucleic acid concatemer. In some embodiments, the nucleic acid concatemer comprises two or more photoreactive nucleotides in the hybridization region. In some embodiments, the two or more photoreactive nucleotides are in the same sequence of the hybridization region. For example, each of the two or more photoreactive nucleotides may be in the 5′ end sequence of the hybridization region. In contrast, the two or more photoreactive nucleotides may not be in the same sequence of the hybridization region. For example, one photoreactive nucleotide may be in the 5′ end sequence of the hybridization region, while a second photoreactive nucleotide may be in the internal sequence of the hybridization region. In some embodiments, the nucleic acid concatemer comprises three, four, five or more photoreactive nucleotides in the hybridization region.

In some aspects, provided herein are methods and compositions for crosslinking a nucleic acid concatemer to a substrate (e.g., a complementary strand of a target nucleic acid). In some embodiments, the nucleic acid concatemer is crosslinked to the complementary strand via a hybridization region comprising a crosslinkable moiety (e.g., at least one photoreactive nucleotide). In some embodiments, the photoreactive nucleotide (e.g., a nucleotide comprising a 3-cyanovinylcarbazole (^{CNV}K) nucleoside) is photo-activated to initiate crosslinking of the nucleic acid concatemer to the complementary strand. In some embodiments, the photoreactive nucleotide (e.g., a nucleotide comprising a pyranocarbazole (^{PC}X) nucleoside) is photo-activated to initiate crosslinking of the nucleic acid concatemer to the complementary strand. In some embodiments, the photoreactive nucleotide (e.g. a nucleotide comprising a psoralen or a psoralen derivative) is photo-activated to initiate crosslinking of the nucleic acid concatemer to the complementary strand. In some embodiments, photo-activated crosslinking comprises exposing the sample to UV irradiation to activate a photoreactive nucleotide.

The nucleic acid concatemer comprising photoreactive nucleotides may be immobilized within the biological sample generally at the location of the target nucleic acid hybridized by the oligonucleotide probe(s), thereby creating a localized nucleic acid concatemer comprising the target nucleic acid. The nucleic acid concatemer may be immobilized within the biological sample by covalent or noncovalent bonding, e.g., by crosslinking mediated by the one or more photoreactive nucleotides. In this manner, the nucleic acid concatemer may be considered to be attached to the complementary strand comprising the target nucleic acid in the biological sample. In some embodiments, by being immobilized to the complementary strand, such as by covalent bonding or cross-linking, the size and spatial relationship of the original target nucleic acids and nucleic acid concatemer is maintained. In some embodiments, by being immobilized to the complementary strand comprising the target nucleic acid, such as by covalent bonding or cross-linking, the nucleic acid concatemers are resistant to movement or unraveling under mechanical stress. In some aspects, the immobilization of the oligonucleotide to the complementary strand, such as by covalent bonding or cross-linking, maintains the position of the growing nucleic acid concatemer in the sample during rolling circle amplification.

The photoreactive nucleotides may be photo-activated by UV light, such as a 350-400 nm wavelength of light, to photo-activate and crosslink the nucleic acid concatemer to the complementary strand. In some embodiments, the photoreactive nucleotides are crosslinked to the complementary strand at a 355 nm wavelength of light. In some embodiments, the purine bases of the complementary strand are unreactive to photo-activated crosslinking. In some embodiments, the pyrimidine bases of the complementary strand are reactive to photo-activated crosslinking. In some embodiments, the photo-activating step is performed after the nucleic acid concatemer is generated. For example, the photo-activating step may be performed following amplification (e.g., RCA) of a target nucleic acid. In some embodiments, the photo-activating step is performed before the nucleic acid concatemer is generated. For example, an oligonucleotide probe comprising a hybridization region, wherein the hybridization region comprises a photoreactive nucleic acid, hybridizes to a complementary strand of the target nucleic acid. In this example, the photoreactive nucleic acid may be photo-activated to crosslink the oligonucleotide probe to the complementary region prior to amplification of the target nucleic acid (e.g., prior to formation of a nucleic acid concatemer). In some embodiments, the nucleic acid concatemer is generated using an oligonucleotide (e.g., as a primer) comprising a photoreactive nucleotide such that amplification (e.g., RCA) can proceed while the photoreactive nucleic acid is photo-activated and crosslinked to the complementary strand during RCA. Alternatively, the photo-activating step may be performed during amplification of the target nucleic acid (e.g., performed as the nucleic acid concatemer is being generated).

The photo-activated crosslinking step may be optimized to prevent DNA damage. In some embodiments, the photo-activated crosslinking does not cause significant DNA damage. In some embodiments, the photo-activated crosslinking of the nucleic acid concatemer or the oligonucleotide hybridization region to the complementary strand increases the UV melting temperature of the duplex compared to prior to the crosslinking. In some embodiments, the UV melting temperature is increased by about 30° C. per photoreactive nucleotide in the hybridization region. This increase in melting temperature allows the nucleic acid concatemer to be immobilized to the complementary strand, thereby maintaining spatial fidelity during downstream analyses.

In some embodiments, the photo-activated crosslinking is reversible. In some embodiments, the photo-activated crosslinking is partially reversible. In some embodiments, the photo-activated crosslinking is completely reversible. In some embodiments, the reverse crosslinking comprises exposing the sample to UV light, such as between about 310 nm and 315 nm wavelength of light. In some embodiments, the reverse crosslinking comprises exposing the sample to 312 nm wavelength of light. In some embodiments, the reverse crosslinking comprises about 3 minutes.

The photo-activated crosslinking maintains spatial orientation of the nucleic acid concatemer relative to the target nucleic acids the presence of denaturing agents. In some embodiments, the method further comprises processing the biological sample comprising the crosslinked nucleic acid concatemer. In some embodiments, the processing comprises subjecting the biological sample comprising the crosslinked nucleic acid concatemer to a denaturing condition. In some embodiments, the denaturing condition comprises a contacting the biological sample with a denaturing agent and/or heating the biological sample. In some embodiments, the denaturing agent comprises formamide, guanidine, sodium salicylate, dimethyl sulfoxide (DMSO), ethylene carbonate, propylene glycol, or urea. In some embodiments, the denaturing agent comprises formamide. In some embodiments, the denaturing comprises heating the biological sample to disrupt base pairing between the nucleic acid concatemer and the complementary strand. In some embodiments, the biological sample is denatured upon heating above about 80° C. In some embodiments, the biological sample is subjected to repeated cycles of washes that may include denaturing conditions.

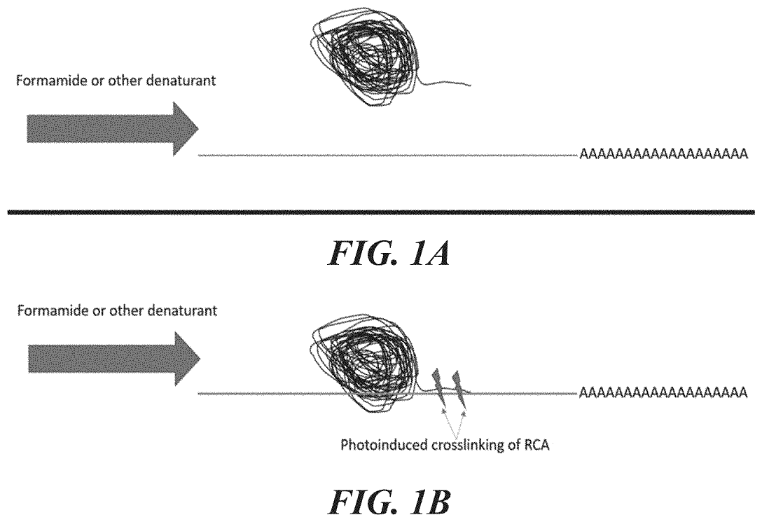

In some embodiments, the nucleic acid concatemer crosslinked to the complementary strand remains associated with the complementary strand during the processing of the biological sample, e.g., under denaturing conditions, such as shown in FIG. 1B. For example, the increased UV melting temperature of the duplex in the nucleic acid concatemer compared to prior to the crosslinking is resistant to the denaturing agents. In some embodiments, a reference nucleic acid concatemer not comprising a photoreactive nucleotide (e.g., a reference nucleic acid concatemer not crosslinked to the complementary strand) is dissociated from the complementary strand during processing of the biological sample, e.g., under denaturing conditions, such as shown in FIG. 1A.

### V. Detection and Analysis

In some aspects, the provided methods involve analyzing, e.g., detecting or determining, one or more sequences present in the target nucleic acid and/or in the nucleic acid concatemers comprising at least one photoreactive nucleotide as described herein. In some embodiments, the detecting comprises hybridizing one or more detectably labeled probes to the nucleic acid concatemer, or via hybridization to adaptor probes that hybridize to the nucleic acid concatemer). In some embodiments, the analysis comprises determining the sequence of all or a portion of the nucleic acid concatemer (e.g., a barcode sequence or a complement thereof), wherein the sequence is indicative of a sequence of the target nucleic acid.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization in a FISH-type assay, sequencing by hybridization).

In some embodiments, the methods comprise sequencing all or a portion of the nucleic acid concatemer or detecting a sequence of the nucleic acid concatemer, such as one or more barcode sequences present in the nucleic acid concatemer. In some embodiments, the sequence of the nucleic acid concatemer, or barcode thereof, is indicative of a sequence of the target nucleic acid to which the nucleic acid concatemer is hybridized. In some embodiments, the analysis and/or sequence determination comprises sequencing all or a portion of the nucleic acid concatemer and/or in situ hybridization to the nucleic acid concatemer. In some embodiments, the sequencing step involves sequencing by hybridization, sequencing by ligation, sequencing by synthesis, sequencing by binding, and/or fluorescent in situ sequencing (FISSEQ), hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction. In some embodiments, the detection or determination comprises hybridizing to the first overhang a detection oligonucleotide labeled with a fluorophore, an isotope, a mass tag, or a combination thereof. In some embodiments, the detection or determination comprises imaging the probe hybridized to the target nucleic acid (e.g., imaging one or more detectably labeled probes hybridized thereto). In some embodiments, the target nucleic acid is an mRNA in a tissue sample, and the detection or determination is performed when the target nucleic acid and/or the amplification product is in situ in the tissue sample. In some embodiments, the target nucleic acid is an amplification product (e.g., a rolling circle amplification product/nucleic acid concatemer).

In some aspects, the provided methods comprise imaging the probe hybridized to the nucleic acid concatemer, for example, via binding of the secondary probe (e.g., a detection probe) and detecting the detectable label. In some embodiments, the detection probe comprises a detectable label that can be measured and quantitated. The label or detectable label can comprise a directly or indirectly detectable moiety that is associated with (e.g., conjugated to) a molecule to be detected, e.g., a secondary probe that is a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. Background fluorescence can include autofluorescence (that can arise from a variety of sources, including aldehyde fixation, extracellular matrix components, red blood cells, lipofuscin, and the like), as opposed to the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label can be used to detect one or more nucleic acid concatemer(s) crosslinked to the complementary strand of the target nucleic acid described herein. In some embodiments, the methods involve incubating the detectable probe containing the detectable label with the sample, washing unbound detectable probe, and detecting the label, e.g., by imaging. In some embodiments, the nucleic acid concatemer(s) remain crosslinked to the target nucleic acid during the washing and detecting steps.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Labeling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are herein incorporated by reference in their entireties. In some embodiments, a fluorescent label comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-!2-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods are known for custom synthesis of nucleotides having other fluorophores (See, Henegariu et al. (2000) Nature Biotechnol. 18:345).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a oligonucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and 5,073,562, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECS™), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequencing can be performed in situ. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in US 2016/0024555, US 2019/0194709, and in U.S. Pat. Nos. 10,138,509, 10,494,662 and 10,179,932, all of which are herein incorporated by reference in their entireties. Exemplary techniques for in situ sequencing or in in situ sequence detection comprise, but are not limited to, STARmap (described for example in Wang et al., (2018) Science, 361(6499) 5691), MERFISH (described for example in Moffitt, (2016) Methods in Enzymology, 572, 1-49), hybridization-based in situ sequencing (HybISS) (described for example in Gyllborg et al., Nucleic Acids Res (2020) 48(19):e112, and FISSEQ (described for example in US 2019/0032121, the content is herein incorporated by reference in its entirety).

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing can be performed by sequential fluorescence hybridization (e.g., sequencing by hybridization). Sequential fluorescence hybridization can involve sequential hybridization of detection probes comprising an oligonucleotide and a detectable label.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, all of which are herein incorporated by reference in their entireties.

In some embodiments, the barcodes of the detection probes are targeted by detectably labeled secondary probe oligonucleotides, such as fluorescently labeled oligonucleotides. In some embodiments, one or more decoding schemes are used to decode the signals, such as fluorescence, for sequence determination. In any of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); Gyllborg et al., Nucleic Acids Res (2020) 48(19):e112; U.S. Pat. No. 10,457,980 B2; US 2016/0369329 A1; WO 2018/026873 A1; and US 2017/0220733 A1, all of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are herein incorporated by reference in their entireties.

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

### VI. Compositions, Kits, and Systems

In some embodiments, disclosed herein is a composition that comprises a complex containing a target nucleic acid, an oligonucleotide probe comprising a primer and a hybridization region, and a circular probe or circularizable probe or probe set, e.g., any of the target nucleic acids, oligonucleotide probes, and circular probes described in Section III. In some embodiments, the complex further comprises a secondary probe (e.g., a detection probe), e.g., as described in Section V. In some embodiments, the hybridization region of the oligonucleotide probe or nucleic acid concatemer resulting from amplification of the target nucleic acid comprises modified nucleotides (e.g., photoreactive nucleotides), such that the photoreactive nucleotides are attached to the hybridization region. In some embodiments, the composition further comprises one or more modified nucleotides, e.g., any of the photoreactive nucleotides described in Section IV.

Also provided herein are kits, for example comprising one or more oligonucleotides, e.g., any described in Section III, and instructions for performing the methods provided herein. In some embodiments, the kits further comprise one or more reagents for performing the methods provided herein (e.g., one or more photoreactive nucleotides, such as any of the photoreactive nucleotides described in Section IV). In some embodiments, the kits further comprise one or more reagents required for one or more steps comprising hybridization, ligation, extension, detection, sequencing, and/or sample preparation as described herein. In some embodiments, the kit further comprises a target nucleic acid, e.g., any described in Section III. In some embodiments, any or all of the oligonucleotides are DNA molecules. In some embodiments, the target nucleic acid is a messenger RNA molecule. In some embodiments, the target nucleic acid is a probe (e.g., a padlock probe) or an amplification product thereof (e.g., a rolling circle amplification product, such as a nucleic acid concatemer). The various components of the kit may be present in separate containers or certain compatible components may be precombined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, photoreactive nucleotides, and reagents for additional assays.

### VII. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to identify or detect single nucleotides of interest in target nucleic acids. In some aspects, the provided embodiments can be used to crosslink the nucleic acid concatemers via photoreactive nucleotides, e.g., to a complementary strand, to increase the stability of the nucleic acid concatemers probe in situ.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VIII. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, i.e., where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif.; HeliScope™ by Helicos Biosciences Corporation, Cambridge, Ma.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods. “Multiplexing” or “multiplex assay” herein may refer to an assay or other analytical method in which the presence and/or amount of multiple targets, e.g., multiple nucleic acid target sequences, can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLE

The following example is included for illustrative purposes only and is not intended to limit the scope of the present disclosure.

### Example 1: Photo-Crosslinking Nucleic Acid Concatemers Increases the Fidelity of In Situ Analyses

This example demonstrates a method for analyzing a biological sample. In particular, this example demonstrates the covalent anchoring of a rolling circle amplification (RCA) product to a target nucleic acid to enhance spatial fidelity of the resulting RCA.

A biological sample (e.g., a processed or cleared biological sample, a tissue sample, a sample embedded in a hydrogel, etc.) is contacted with a circular probe or circularizable probe or probe set, and with an oligonucleotide. The oligonucleotide comprises a primer sequence that hybridizes to the circular probe or circularizable probe or probe set. The oligonucleotide additionally comprises a hybridization region that hybridizes to a target nucleic acid in the biological sample. The hybridization region of the oligonucleotide comprises at least one photoreactive nucleotide, which is capable of photo-crosslinking to pyrimidines on a complementary strand (e.g., RNA, DNA, or cDNA) in the target nucleic acid the biological sample. In some embodiments, the photoreactive nucleotide comprises a 3-cyanovinylcarbazole (^{CNV}K) nucleoside, 3-cyanovinylcarbazole modified D-threoninol (^{CNV}D), or 3-cyanovinylcarbazole phosphoramidite. In some embodiments, the photonucleotide comprises a pyranocarbazole (^{PC}X) nucleoside, pyranocarbazole modified D-threoninol (^{PCX}D). or a pyranocarbazole phosphoramidite

The oligonucleotide may comprise a sequence that hybridizes to the circular probe or circularizable probe or probe set, and/or the oligonucleotide may be ligated to a primer that hybridizes to the circular probe or circularizable probe or probe set. Thus, the oligonucleotide or the ligated oligonucleotide-prime can be used to prime an RCA using the circular or circularized probe as a template. Modified nucleotides such as amine-modified nucleotides can be incorporated into the RCA product.

The photo-activating step may be performed prior to, during, and/or after the RCA generation. In one example, as described above, photo-activation of the photoreactive nucleotide occurs prior to RCA. Alternatively, an RCA product is first generated in the biological sample, and upon generation, the RCA product is subsequently crosslinked to the target nucleic acid via photo-activation of the photoreactive nucleotide.

The photoreactive nucleotide is photo-activated to react with a nucleotide on the complementary strand in the target nucleic acid, thereby crosslinking the oligonucleotide to the target nucleic acid. Photo-activation is performed by inducing rapid photo-crosslinking using a specific wavelength of light. The wavelength of light used for photo-activation does not cause significant DNA damage, and does not interfere with the wavelengths used to excite any fluorophores used during downstream analysis (e.g., detection/decoding). In some embodiments, the photo-activation is performed using a 350-400 nm wavelength of light. Once cross-linked, the UV melting temperature of the duplex is raised by around 30° C. per photoreactive nucleotide, relative to the duplex before irradiation, thus stabilizing the interaction between the nucleic acid concatemer and the complementary strand.

In some experiments, the primer sequence of the oligonucleotide is used as a primer and the circular probe, or a circularized probe generated from the circularizable probe or probe set, is used as a template. The RCA product therefore comprises the oligonucleotide, or a portion thereof, crosslinked to the target nucleic acid via the photoreactive nucleotide. The RCA product further comprises a barcode which may be detected.

In some experiments, the biological sample is contacted with an intermediate probe that hybridizes to the RCA product. The intermediate probe further comprises one or more binding regions for fluorescently-labeled probes. Once a signal associated with the RCA product is detected in one probe hybridization cycle, the intermediate probe and/or fluorescently-labeled probes can be dehybridized from the RCA product (e.g., by washing). The RCA product remains crosslinked to the nucleic acid in the biological sample via the photoreactive nucleotide during the dehybridization process. Multiple probe hybridization and dehybridization cycles can be performed to allow for decoding of the barcode sequence in the RCA product.

This method maintains the association of the RCA product in the sample with its respective target nucleic acid (e.g., via the complementary strand), without the additional complexity of a hydrogel. Increasing the spatial fidelity of each RCA product in a sample with its unique target prevents the confusion of algorithms during downstream analysis including decoding.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method for processing a biological sample, comprising:
(a) generating a nucleic acid concatemer in the biological sample, wherein the nucleic acid concatemer comprises a photoreactive nucleotide in a hybridization region hybridized to a complementary strand; and
(b) photo-activating the photoreactive nucleotide to react with a nucleotide in the complementary strand,
thereby crosslinking the nucleic acid concatemer to the complementary strand.

2-5. (canceled)

6. The method of claim 1, wherein the nucleic acid concatemer is a rolling circle amplification product.

7-11. (canceled)

12. The method of claim 1, wherein the photoreactive nucleotide is a modified nucleotide comprising a psoralen or a psoralen derivative.

13. (canceled)

14. The method of claim 1, wherein the photoreactive nucleotide is a modified nucleotide comprising a vinylcarbazone-based moiety.

15. The method of claim 14, wherein the vinylcarbazone-based moiety comprises a 3-cyanovinylcarbazole (^{CNV}K) nucleoside, 3-cyanovinylcarbazole modified D-threoninol (^{CNV}D), a pyranocarbazole nucleoside (^{PC}X) or a pyranocarbazole modified D-threoninol (^{PCX}D).

16-17. (canceled)

18. The method of claim 1, wherein the nucleic acid concatemer comprises two or more photoreactive nucleotides in the hybridization region.

19-25. (canceled)

26. The method of claim 1, wherein the complementary strand is in a cellular RNA in the biological sample.

27-30. (canceled)

31. The method of claim 1, further comprising providing a circular probe or circularizable probe or probe set and an oligonucleotide comprising the hybridization region before the nucleic acid concatemer is generated in step (a), wherein the oligonucleotide and the circular probe or circularizable probe or probe set hybridize to adjacent regions in the complementary strand,
wherein the oligonucleotide comprises a primer region that hybridizes to the circular probe or circularizable probe or probe set, whereby the nucleic acid concatemer is generated using the oligonucleotide as a primer and the circular probe or circularizable probe or probe set as a template.

32-36. (canceled)

37. The method of claim 31, wherein the oligonucleotide comprises one or more universal bases.

38. The method of claim 37, wherein the photoreactive nucleotide comprises the universal base.

39. The method of claim 37, wherein the oligonucleotide comprises two or more photoreactive nucleotides in the hybridization region, and wherein the photoreactive nucleotides comprise the universal bases.

40. The method of claim 37, wherein the one or more universal bases comprise a pseudouridine and/or an inosine.

41-42. (canceled)

43. The method of claim 37, wherein the hybridization region of the oligonucleotide hybridizes to the complementary strand non-specifically.

44. The method of claim 31, wherein the primer region is a common primer region complementary to a plurality of different circular or circularizable probes or probe sets that are contacted with the biological sample.

45. The method of claim 31, further comprising ligating the circularizable probe or probe set to generate a circularized template for rolling circle amplification to generate the nucleic acid concatemer, wherein the ligating is prior to, during, or after the photo-activating.

46-55. (canceled)

56. The method of claim 1, further comprising detecting the nucleic acid concatemer at a location in the biological sample.

57. The method of claim 56, wherein the detecting comprises:
contacting the biological sample with one or more detectably-labeled probes that directly or indirectly hybridize to the nucleic acid concatemer, and
dehybridizing the one or more detectably-labeled probes from the nucleic acid concatemer.

58. (canceled)

59. The method of claim 56, wherein the detecting comprises:
contacting the biological sample with one or more intermediate probes that directly or indirectly hybridize to the nucleic acid concatemer, wherein the one or more intermediate probes are detectable using one or more detectably-labeled probes, and
dehybridizing the one or more intermediate probes and/or the one or more detectably-labeled probes from the nucleic acid concatemer.

60-61. (canceled)

62. A method for analyzing a biological sample, comprising:
(a) contacting the biological sample with:
a circular probe or circularizable probe or probe set, and
an oligonucleotide comprising (i) a hybridization region that hybridizes to a target nucleic acid in the biological sample and (ii) a primer sequence that hybridizes to the circular probe or circularizable probe or probe set,
wherein the hybridization region comprises a photoreactive nucleotide;
(b) photo-activating the photoreactive nucleotide to react with a nucleotide in the target nucleic acid, thereby crosslinking the oligonucleotide to the target nucleic acid;
(c) generating a rolling circle amplification (RCA) product in the biological sample, using the primer sequence as a primer and the circular probe or a circularized probe generated from the circularizable probe or probe set as a template, wherein the RCA product comprises the oligonucleotide or a portion thereof crosslinked to the target nucleic acid;
(d) contacting the biological sample with a detection probe that hybridizes to the RCA product; and
(e) dehybridizing the detection probe from the RCA product while the RCA product remains crosslinked to the target nucleic acid in the biological sample.

63-87. (canceled)

88. The method of claim 1, wherein the method comprises contacting the biological sample with a plurality of different circular or circularizable probes or probe sets that hybridize to a plurality of different complementary strands in the biological sample, and
performing rolling circle amplification to generate a plurality of different nucleic acid concatemers from the circular or circularizable probes or probe sets in the biological sample,
wherein the nucleic acid concatemers comprise a photoreactive nucleotide in a hybridization region hybridized to the corresponding complementary strand; and
photo-activating the photoreactive nucleotides to react with nucleotides in the complementary strand,
thereby crosslinking the nucleic acid concatemers to the corresponding complementary strands.
